(19) United States
(12) Patent Application Publication
(10) Pub. No.: US 2023/0032180 A1
(43) Pub. Date: Feb. 2, 2023

(54) METHOD AND SYSTEM FOR EMPOWERING CANCER PATIENT(S)

(71) Applicant: Innoplexus AG, Eschborn (DE)

(72) Inventors: Dhiraj Jadhao, Pune (IN); Chiara Thanner, Frankfurt (DE)

(73) Assignee: Innoplexus AG, Eschborn (DE)

(21) Appl. No.: 17/388,661

(22) Filed: Jul. 29, 2021

(51) Int. Cl. G16H 40/20; G16H 10/60; G16H 50/70; G16H 10/20; G16H 70/20

(52) U.S. Cl. CPC G16H 40/20; G16H 10/60; G16H 50/70; G16H 10/20; G16H 70/20

(57) ABSTRACT

There is disclosed a system for providing information to a plurality of cancer patients, the system including a server arrangement communicably coupled to a database arrangement comprising a plurality of records and a user device, wherein the server arrangement is configured to create profile for a plurality of patients by receiving information from the user device, discover available treatment options for the created profile, list recruiting clinical trials for the created profile, list relevant oncologists for the created profile for at least one consultation and map the created profile against pre-existing profiles and connecting the determined profile and the pre-existing profiles for experience sharing purposes.

### TECHNICAL FIELD

The present disclosure relates generally to system for providing various information to patients, and more specifically, to system and method for providing information related to treatment protocols, recruiting clinical trials and relevant oncologists to a plurality of cancer patients.

### BACKGROUND

Cancer, a leading fatal disease, features an abnormal mass of malignant tissue resulting from excessive cell division. Cancer cells proliferate in defiance of normal restraints on cell growth, and invade and colonize territories normally reserved for other cells. Conventional treatment protocols for cancer include chemotherapy, surgery, radiation, and combinations of these treatments.

Moreover, a patient going through any conventional or experimental treatment protocol of cancer has to face lots of physical and mental trauma/stress. The biggest reason of facing physical and mental trauma is unavailability of correct and relevant information to various patients.

Conventionally, the information available on the internet is scattered in bites and pieces, also, the accuracy of the available information is always questionable as it is not validated. Thus, a person facing issues related to cancer always face challenge in opting a treatment protocol for cancer.

Moreover, the experience of cancer survivors and patients going through any treatment protocol is very useful for new patients and helps patients to reduce mental trauma or stress.

Therefore, in light of the foregoing discussion, there exists a need to overcome the aforementioned drawbacks associated with existing information retrieval system for cancer patients.

### SUMMARY

The present disclosure seeks to provide a system for providing information to a plurality of cancer patients. The present disclosure also seeks to provide a method of providing information to a plurality of cancer patients. The present disclosure seeks to provide a solution to the existing problem of unmanageable, unstructured, time consuming and inefficient techniques of information retrieval system for cancer patients.

An aim of the present disclosure is to provide a solution that overcomes at least partially the problems encountered in prior art, and provides processing and time-efficient method of information retrieval for cancer patients.

In one aspect, the present disclosure provides a system for providing information to a plurality of cancer patients, the system comprising a server arrangement communicably coupled to a database arrangement comprising a plurality of records and a user device, wherein the server arrangement is configured to:
create profile for a plurality of patients by receiving information from the user device;
discover available treatment options for the created profile;
list recruiting clinical trials for the created profile;
list relevant oncologists for the created profile for at least one consultation; and
   map the created profile against pre-existing profiles and connecting the determined profile and the pre-existing profiles for experience sharing purposes.

Embodiments of the disclosure are advantageous in terms of providing an easy-to-use information retrieval system for cancer patients. Also, the system empowers patient(s) with information to navigate cancer journey. The system provides accurate and current information on therapies, clinical trials (existing and upcoming), and experts, based on your cancer profile and details entered in the system. Moreover, information retrieval system is updated in real-time so that the most recently approved therapies and launched clinical trials are at patient's fingertips.

Optionally, the information received for the user device includes bibliographic information, cancer type, pre-existing conditions and geographical location of the user.

Optionally, the available treatment options include at least one of a conventional treatment protocols and off-label treatment protocols.

Optionally, the list of the recruiting clinical trials is based on at least one of inclusion and exclusion criteria.

Optionally, wherein the system is further configured to save the relevant oncologists as favourites.

In another aspect, the present disclosure provides a method for providing information to a plurality of cancer patients, the method comprising:
creating profile of the plurality of patients;
discovering available treatment options for the created profile;
listing recruiting clinical trials for the created profile;
listing relevant oncologists for the created profile for at least one consultation; and
   mapping the created profile against pre-existing profiles and connecting the determined profile and the pre-existing profiles for experience sharing purposes.

Optionally, creating profile of the plurality of patients includes receiving bibliographic information, cancer type, pre-existing conditions and geographical location of a patient.

Optionally, the available treatment options include at least one of a conventional treatment protocols and off-label treatment protocols.

Optionally, listing of the recruiting clinical trials is based on at least one of inclusion and exclusion criteria.

Optionally, the method further includes saving the relevant oncologists as favourites.

Embodiments of the present disclosure substantially eliminate or at least partially address the aforementioned problems in the prior art, and provides a manageable and efficient method for providing information to a plurality of cancer patients.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary embodiments of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is an illustration of a network environment in which a system for providing information to a plurality of cancer patients is implemented, in accordance with an embodiment of the present disclosure; and

FIG. 2 is an illustration of steps of a method for providing information to a plurality of cancer patients, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item to which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognise that other embodiments for carrying out or practising the present disclosure are also possible.

In one aspect, the present disclosure provides a system for providing information to a plurality of cancer patients, the system comprising a server arrangement communicably coupled to a database arrangement comprising a plurality of records and a user device, wherein the server arrangement is configured to:
create profile for a plurality of patients by receiving information from the user device;
discover available treatment options for the created profile;
list recruiting clinical trials for the created profile;
list relevant oncologists for the created profile for at least one consultation; and
   map the created profile against pre-existing profiles and connecting the determined profile and the pre-existing profiles for experience sharing purposes.

In another aspect, the present disclosure provides a method for providing information to a plurality of cancer patients, the method comprising:
creating profile of the plurality of patients;
discovering available treatment options for the created profile;
listing recruiting clinical trials for the created profile;
listing relevant oncologists for the created profile for at least one consultation; and
   mapping the created profile against pre-existing profiles and connecting the determined profile and the pre-existing profiles for experience sharing purposes.

The present disclosure provides a system and method of providing information to a plurality of cancer patients that is efficient in terms of time and processing power required for use thereof. The system and method of the present disclosure enable disambiguation of information relating to cancer treatment protocols, therapies, clinical trials (existing and upcoming), and experts, thereby allowing an increased amount of information to be available for a cancer patient. Furthermore, the system significantly reduces entity recognition errors, ambiguous references. The system described herein de-duplicates repetitive information belonging to the same entity, thereby significantly reducing sizes of datasets and processing power required for processing thereof. Additionally, the method described herein does not require human intervention for functioning thereof. Furthermore, the method exhibits a very low computational (namely, processing) and time complexity.

Moreover, embodiments of the disclosure are advantageous in terms of providing an easy-to-use information retrieval system for cancer patients. Also, the system empowers patient(s) with information to navigate cancer journey. The system provides accurate and current information on therapies, clinical trials (existing and so upcoming), and experts, based on your cancer profile and details entered in the system. Moreover, information retrieval system is updated in real-time so that the most recently approved therapies and launched clinical trials are at patient's fingertips.

The system comprises a server arrangement. Herein, the term “server arrangement” refers to a structure and/or module that include programmable and/or non-programmable components configured to store, process and/or share information. Optionally, the server arrangement includes any arrangement of physical or virtual computational entities capable of enhancing information to perform various computational tasks. Furthermore, it should be appreciated that the server may be both single hardware server and/or plurality of hardware servers operating in a parallel or distributed architecture. In an example, the server may include components such as memory, a processor, a network adapter and the like, to store, process and/or share information with other computing components, such as user device/user equipment. Optionally, the server is implemented as a computer program that provides various services (such as database service) to other devices, modules or apparatus.

The server arrangement is communicably coupled to a database arrangement. Herein, the term “database arrangement” refers to an organized body of digital information, regardless of the manner in which the data or the organized body thereof is represented. Optionally, the database may be hardware, software, firmware and/or any combination thereof. For example, the organized body of related data may be in the form of a table, a map, a grid, a packet, a datagram, a file, a document, a list or in any other form. The database includes any data storage software and systems, such as, for example, a relational database like IBM DB2 and Oracle 9.

The database arrangement stores a plurality of records/documents. Herein, the term “record(s)” or “documents” refers to electronic documents comprising information stored in a digital format. Notably, the information is recorded as a data type. Some examples of various data types are text data, tabular data, image data, and so forth. Thus, documents may be in any suitable file formats depending upon the data type in which the information is recorded. The document may be a research paper, a technical publication, a clinical trial, a study result, medical journals, patent documents and the like. Notably, the plurality of documents may be domain specific.

The server arrangement is configured to generate an entity network by parsing the plurality of documents, wherein the entity network comprises a plurality of entities and their relationships, the plurality of entities comprising at least: document entities, name entities and topic entities. Herein, the term “entity” refers to an attribute of a document that provides characteristic information about the document. Examples of such characteristic information may include, but is not limited to, name of an author of the document, names of persons mentioned in the document, a unique identifier of the document, a topic to which the document belongs, content of the document, title of the document, publication organization from where the document originated, location of the publication organization. Therefore, attributes representing such characteristic information are extracted from the plurality of documents by parsing thereof and included in the entity network as entities. Specifically, parsing refers to analysing a document and determining syntactic roles of the content in the document using syntax analysis. Such syntactic analysis provides segregation of content in the document based on content type (such as cancer type, location and stage) and allow isolation of key information from the document. Furthermore, the server arrangement may parse metadata related to the document. Specifically, the metadata related to the document comprises tabulated information that is principal to the document.

The server arrangement and the database arrangement are communicably coupled to a user devices. Herein, the term “user device(s)” refers to a computing device and/or portable computing device. The computing device and/or portable computing device may include but not limited to a mobile device, a tablet and a personal computer.

In an embodiment, the information received for the user device includes bibliographic information, cancer type, pre-existing conditions and geographical location of the user. The bibliographic information may include but not limited to name, age, sex, height, weight and any other relevant information. The pre-existing conditions may include details related to existing medical conditions of the patient such as heart condition, blood pressure or any other information related to health condition of the patient(s).

The server arrangement is configured to generate an entity network by parsing the plurality of documents, wherein the entity network comprises a plurality of entities and their relationships, the plurality of entities comprising at least: document entities, name entities and topic entities. Herein, the term “entity” refers to an attribute of a document that provides characteristic information about the document. Specifically, parsing refers to analysing a document and determining syntactic roles of the content in the document using syntax analysis. Such syntactic analysis provides segregation of content in the document based on content type and allow isolation of key information from the document. Furthermore, the server arrangement may parse metadata related to the document. Specifically, the metadata related to the document comprises tabulated information that is principal to the document.

Optionally, extracting entities from the documents comprises cleaning and/or translating the documents. Specifically, cleaning the documents refers to removal of unnecessary comments, annotations, symbols, images and/or a combination thereof. Consequently, the server arrangement extracts only relevant information from the existing data sources. Moreover, translating the documents refers to conversion thereof to a machine-readable form. Beneficially, cleaning and/or translating the documents reduce processing complexity thereof. Additionally, cleaning and/or translating the documents also reduce processing time for identifying information relating to the entity. Optionally, a dedicated and adaptive subroutine may extract the information relating to the entities.

Optionally, the server arrangement is configured to determine, a relationship score of at least one relationship between a document entity and a name entity, based on classifiers of name entity that include at least one of: authored, mentioned. As mentioned previously, a name entity is representative of information relating to persons associated with the document.

Furthermore, the server arrangement is configured to identify relationships between the topic entities and at least one document entity. Specifically, relationships are identified between the document entities and the topic entities that are identified from those document entities. In an example, from a document represented by document entity ‘A’, identified topic entities are ‘artificial intelligence’ and ‘DNA sequencing’. Therefore, relationships between the topic entities, ‘artificial intelligence’ and ‘DNA sequencing’, and the document entity ‘A’ are established. Such identification of relationships is performed for every topic entity that is identified in each document from the plurality of documents.

The server arrangement is configured to determine a relevance score of at least one document entity based on relationships thereof with the name entities and the topic entities, and the importance score of each of the name entities using link analysis algorithm. Herein, the term “relevance score” as used herein the present disclosure relates to a measure of degree of relevance or significance of at least one document entity in the entity network.

In an embodiment, based on the relevance score the user may contact with cancer survivor and other patients undergoing any of the cancer treatment protocol via the chat function of the system.

In another embodiment, the available treatment options may include but not limited to a conventional treatment protocols and off-label treatment protocols.

In yet another embodiment, the available treatment options may include at least one of a conventional treatment protocols and off-label treatment protocols.

In an embodiment, the list of the recruiting clinical trials may be based on at least one of inclusion and exclusion criteria.

In another embodiment, the system is further configured to save the relevant oncologists as favourites for quick consultation process. The server arrangement enables the user to take a first opinion for the relevant oncologists and to take a second opinion form another relevant oncologists.

The present disclosure also relates to the method as described above. Various embodiments and variants disclosed above apply mutatis mutandis to the method.

Optionally, creating profile of the plurality of patients includes receiving bibliographic information, cancer type, pre-existing conditions and geographical location of a patient.

Optionally, the available treatment options include at least one of a conventional treatment protocols and off-label treatment protocols.

Optionally, listing of the recruiting clinical trials is based on at least one of inclusion and exclusion criteria.

Optionally, the method further includes saving the relevant oncologists as favourites.

### DETAILED DESCRIPTION OF THE DRAWINGS

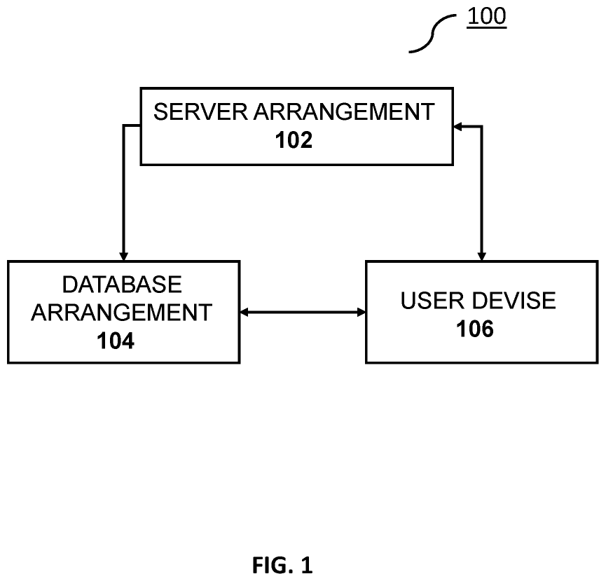

Referring to FIG. 1, there is shown a network environment 100 in which a system for providing information to a plurality of cancer patients is implemented, in accordance with an embodiment of the present disclosure. The system comprises a server arrangement 102 communicably coupled to a database arrangement 104 comprising a plurality of records and a user device 106, wherein the server arrangement 102 is configured to:
create profile for a plurality of patients by receiving information from the user device;
discover available treatment options for the created profile;
list recruiting clinical trials for the created profile;
list relevant oncologists for the created profile for at least one consultation; and
   map the created profile against pre-existing profiles and connecting the determined profile and the pre-existing profiles for experience sharing purposes.

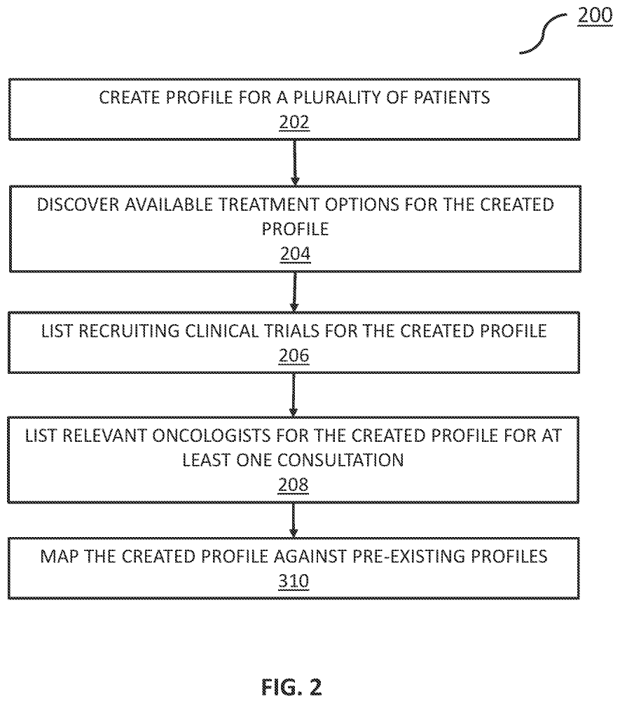

Referring to FIG. 2, illustrated are steps of a method 200 for providing information to a plurality of cancer patients, in accordance with an embodiment of the present disclosure. The method is depicted as a collection of steps in a logical flow diagram, which represents a sequence of steps that can be implemented in hardware, software, or a combination thereof, for example as aforementioned. The method 200 is implemented using a system comprising a server arrangement communicably coupled to a database arrangement comprising a plurality of records and a user device. At a step 202, profile for a plurality of patients is created by receiving information from the user device. At a step 204, available treatment options for the created profile are discovered. At a step 206, recruiting clinical trials are listed for the created profile. At a step 208, relevant oncologists for the created profile are listed for at least one consultation. At a step 210, the created profile are mapped against pre-existing profiles and the determined profile and the pre-existing profiles are connected for experience sharing purposes.

The steps 202 to 210 of method 200, are only illustrative and other alternatives can also be provided where one or more steps are added, one or more steps are removed, or one or more steps are provided in a different sequence without departing from the scope of the claims herein.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural where appropriate.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

1. A system for providing information to a plurality of cancer patients, the system comprising a server arrangement communicably coupled to a database arrangement comprising a plurality of records and a user device, wherein the server arrangement is configured to:
create profile for a plurality of patients by receiving information from the user device;
discover available treatment options for the created profile;
list recruiting clinical trials for the created profile;
list relevant oncologists for the created profile for at least one consultation; and
map the created profile against pre-existing profiles and connecting the determined profile and the pre-existing profiles for experience sharing purposes.

2. A system of claim 1, wherein the information received for the user device includes bibliographic information, cancer type, pre-existing conditions and geographical location of the user.

3. A system of claim 1, wherein the available treatment options include at least one of a conventional treatment protocols and off-label treatment protocols.

4. A system of claim 1, wherein the list of the recruiting clinical trials is based on at least one of inclusion and exclusion criteria.

5. A system of claim 1, wherein the system is further configured to save the relevant oncologists as favourites.

6. A method for providing information to a plurality of cancer patients, the method comprising:
creating profile of the plurality of patients;
discovering available treatment options for the created profile;
listing recruiting clinical trials for the created profile;
listing relevant oncologists for the created profile for at least one consultation; and
mapping the created profile against pre-existing profiles and connecting the determined profile and the pre-existing profiles for experience sharing purposes.

7. A method of claim 6, wherein creating profile of the plurality of patients includes receiving bibliographic information, cancer type, pre-existing conditions and geographical location of a patient.

8. A method of claim 6, wherein the available treatment options include at least one of a conventional treatment protocols and off-label treatment protocols.

9. A method of claim 6, wherein listing of the recruiting clinical trials is based on at least one of inclusion and exclusion criteria.

10. A method of claim 6, wherein the method further includes saving the relevant oncologists as favourites.
